(19) United States
(12) Patent Application Publication
(10) Pub. No.: US 2004/0171998 A1
(43) Pub. Date: Sep. 2, 2004

(54) Isolated wound-treatment arrangement

(76) Inventor: Patrick V. Marasco JR., Boxford, MA (US)

Correspondence Address: D. N. HALGREN, 35 Central St, Manchester, MA, 01944, US

(21) Appl. No.: 10/684,960

(22) Filed: Oct. 14, 2003

Related U.S. Application Data
(63) Continuation-in-part of application No. 09/621,636, filed on Jul. 21, 2000, now Pat. No. 6,635,035.

(51) Int. Cl. A61M 35/00

(52) U.S. Cl. 604/290

(57) ABSTRACT

A patient wound treatment arrangement for isolating and enclosing a wound site and minimizing costs and contamination of such treatment to permit pressurized fluid treatment of the wound in the bag. The arrangement comprises an enclosure bag for attachment to a body portion of a patient. The enclosure bag has a first end and a second end, with at least one of the first end or the second ends being open. At least one inlet port snorkel is arranged through a wall of the enclosure bag. A one way valve is arranged on an inner end of each inlet port snorkel to permit a wound treating nozzle to be inserted therethrough.

This invention relates to wound treatment apparatus and methods to provide isolatable wound-treatment arrangements, and is a continuation in part application of my co-pending U.S. patent application Ser. No. 09/621,636 filed 21 Jul. 2000 which is a continuation in part application of co-pending U.S. Pat. No. 6,562,013, which is a continuation in part patent of my U.S. Pat. No. 6,083,209, which is a division of my U.S. Pat. No. 5,898,998, all of which are herein incorporated by reference in their entirety.

### BACKGROUND OF THE INVENTION

1. Field of the Invention

2. Prior Art

Wound cleansing is a significant requirement to establish and promote its healing. Typically proper wound treatment includes a flushing with a pressurized fluid for debridement and elimination of bacteria. This is accomplished by an irrigation fluid, which fluid is delivered to the wound site at a high pressure. It is believed that the higher the pressure of the fluid delivery to the wound the more successful will be the decontamination thereof. Such fluid pressure may be delivered as a pulsed or a continuous flow. The prior art utilizing such a procedure typically required the medical personnel to utilize a splash protective gown, splash resistant gloves, a face mask, goggles or a face mask shield. Obviously, the higher the pressure of the irrigating fluid, the more splatter and splashing would occur about the treatment site. The hospital protocol to be followed after such a wound treatment procedure requires a complete cleansing and re-sterilization of the room in which the wound treatment procedure occurred. Such pulsed/sprayed wound cleansing and irrigation procedures however do obviate the need for whirlpool baths and made those treatment time requirements much shorter.

It is an object of the present invention to overcome the disadvantages of the prior art.

It is a further object of the present invention to provide a wound treatment arrangement which will shorten the procedure of time and minimize the procedure cost per patient and per operating medical personnel.

It is yet a further object of the present invention to provide a body portion cleansing and irrigation apparatus which may be used for a number of successive functions on the same patient during a wound treatment procedure.

It is still yet a further object of the present invention to provide a complete isolation arrangement of the wound of the patient from the caregiver during a debridement or irrigation wound treatment procedure.

It is still yet another object of the present invention to eliminate equipment and extra procedures such as suctioning apparatus to minimize costs and minimize time of the procedures common in the prior art.

It is a further object of the present invention to minimize or eliminate the need for splash shields during treatment of a patient.

It is yet a still further object of the present invention to provide an enclosure bag which also may include a body or body portion bag to separate and isolate the treated portion from the patient's enclosed untreated body portions.

### BRIEF SUMMARY OF THE INVENTION

The present invention comprises an elongated, flexible, clear plastic bag having a first, closed, distalmost end and a second, open or proximalmost end. In a preferred embodiment of the present invention, the distalmost end is wider than the proximal most end. The distalmost end may be paddle shaped or “omega” shaped so as to comfortably receive a hand or a foot therewithin. The proximalmost or open end may have for example, a releasable bag-gripping closure tab or a strip of semi-aggressive adhesive circumferentially disposed on an inner facing annular portion thereon, so as to permit the second or proximalmost end to be snugged and brought into close comfortable sealing contact about the patient's arm or leg or body portion. The material of the enclosure bag is clear plastic so as to enable the patient's body portion to be readily viewed therewithin.

The enclosure bag has an inner surface which is preferably slightly textured so as to provide a non-smooth surface that won't undesirably grip, stick or adhere to the skin of the patient's limb or body portion during entry or during the procedure. The preferred embodiment of the enclosure bag has a snorkel or inlet tube arranged through a portion thereof at several locations on the bag. The snorkel comprises a flexible sheet having an outer end for receipt and passage therethrough of a treatment nozzle. The snorkel or inlet tube has an inner end which is arranged within the enclosure bag. The inner end of the snorkel may be valve-like such as a duckbill type valve, to prevent pressure or treatment fluid from escaping therethrough either during treatment or subsequent to that treatment.

A drainage tube is preferably disposed through a sidewall of theenclosure bag at a lower location thereon, generally opposite to the snorkel inlet tubes. The drainage tube includes an elongated sheath which feeds into a collection chamber. The drainage tube preferably has a one way valve at its uppermost end to prevent reversal of flow from the discharge tube or collection chamber back into the body portion enclosure bag. The collection chamber may be a flexible bag itself, or a holding tank. The collection chamber may also itself have a discharge valve thereon. The drainage tube or sheath may be sealable after a wound treatment procedure has taken place, by tying the elongated drainage tube into a knot or sealing it with a seal clip or wire.

The treatment nozzle has a proximal end which is manipulable by the attending physician. The proximal end of the treatment nozzle may include a trigger or pressure adjustment arrangement to select pulsed or continuous fluid treatment through the nozzle. The proximal end of the treatment nozzle is attached to a fluid pressure supply hose in fluid communication with a fluid source which provides the pressured fluid into the treatment nozzle. The arrangement of the drain tube is preferably on the lowermost side of the enclosure bag, opposite the snorkels or input tubes.

Treatment gas or air may be directed through the spray nozzle simultaneously with the fluid, or may be directed therethrough subsequent to fluid pressure treatment, to help maintain the bag as properly spaced apart and out of contact with the patient's skin. The enclosure bag may also be inflated subsequent to the withdrawal of the nozzle to effect that spacing and separation of the interior surface of the enclosure bag from the patient's wound site and skin. Such an enclosure bag may be left on the patient after initial wound treatment to cover the wound, as a type of wound dressing.

The enclosure bag may also act as an isolation bag in a further embodiment, and be filled with oxygen or other medical treatment fluid/gas. A further embodiment contemplates the bag being filled with a gel for treatment of the wound, or a foam or hydrogel combination for the wound treatment. Examples of such treatment fluids include hydrogel, silvadene cream, iodine based products or hydrogen peroxide.

A flutter valve or duck bill type valve is also contemplated in a further embodiment, within the collection chamber. The collection chamber or bag may have gases, liquids or solidifiers added to it such as glutaraldehyde for sanitation and for forming the waste liquid into a gel.

A further embodiment of the present invention comprises a containment or enclosure bag having an open first or distalmost end and an open second or proximalmost end for use as for example in the field of arthroscopy. Both the proximalmost end and the distalmost end would have a sealable lip thereon for snug attachment to the skin of a patient to permit the viewing and operating on an elbow or a knee or the like, of a patient. Such an enclosure bag may have side-placed snorkel or inlet tube and an upper or alternate side snorkel tube for placement of a spray nozzle through an endoscope through that/those snorkel/s and onto/into a joint of the patient being treated. The snorkel tube would may have a duck bill flap valve on its inner side, so as to prevent pressure of fluid from returning out of the snorkel tube. A collection tube may preferably be arranged at a lowermost pocket in the enclosure bag at a generally midpoint thereof. The collection tube would be attached at its distalend to a collection bag or a chamber. The nozzle would be pressure adjustable for continuous or pulsed provision of a treatment fluid from a treatment fluid reservoir in communication with the proximal end of the nozzle shaft. Thus an arthroscopic procedure would be possible without the need for the typically required protective shields and face mask, drains, tubs and subsequent cleansing required procedures of the prior art, while also providing means to collect samples and debris as needed, from the patient's wound.

A yet further embodiment of the present invention is contemplated for treatment of smaller body portions such as for the pulsed or continuous fluid treatment and irrigation of a patient's eye. Such a treatment would include the use of an enclosure bag having an open first end with a semi aggressive adhesive arranged on its open periphery. Such a peripheral adhesive would be disposed about the wound site or the treatment site, for example, such as an eye. Such an enclosure bag would have a snorkel or inlet tube at a central point in the bag, with a duck bill or flutter bill on the inner side of the enclosure bag. The spray nozzle would be permitted to be received through the snorkel inlet tube to spray to the wound or the eye within or covered by the bag. The enclosure bag would have a drainage tube connected to a collection chamber. Such a drainage tube may be cut and sealed at both sides once that cut or breakage in that tube has been made. The spray nozzle or treatment device would have a fluid pressurized source feeding the proximal end of the handle on the nozzle to permit spraying and manipulation by the attending physician. Subsequent to gravity drainage of the irrigation and treatment fluid from the enclosure bag and the sealing of the drainage tube, and removal of the nozzle from the snorkel, the enclosure bag may be left on the treatment site for subsequent use with further medical treatment fluids, medicaments or wound or eye protection.

A further embodiment of the present invention is contemplated as a body enclosure bag wherein an “entire” patient is being treated for a hazardous material contamination. Such a body containment bag would have a plurality of snorkel inlet tubes or ports for the application of treatment gases for the patient therewithin as well as at least one lowermost drainage tube for the passage of the hazardous material into a closed chamber. In such an instance, at least one of those snorkels or inlet tubes may be utilized to provide a constant pressure of treatment gas to keep the enclosure bag away from the surface or skin of the patient therewithin. One or more of the other snorkel inlet tubes may be utilized for the injection of a treatment spray or fluid by the attending medical personnel for the cleansing of the patient, the patient's wounds or washing of hazardous contamination thereof and subsequent closed system collection of that collected hazardous material into the collection chamber. Such treatment may be utilized for burn victims, for patients in nursing homes, for treatment of animals by veterinarians, by ambulance personnel and by military personnel or home care personnel.

Such procedures of utilization of the present invention as an enclosure bag eliminates contamination for the medical personnel attending the patient. It eliminates the whirlpool or sitzbath and is a safer way to do pulse or mechanical irrigation of a wound. Those snorkels or tubes provide the access ports for the various spray nozzles and also may act as handles to permit the gripping thereof and manually keep the bag spaced from the patient's skin if pressure within the bag is insufficient or undesired. In a further preferred embodiment, an outer skeletal frame or support wire of a generally U-shaped configuration may be utilized over the outer surface of the enclosure bag to connectively engage one or more of the snorkel access ports to permit the spacing and support the attached enclosure bag and effect that non-pressurized (or assist the pressurized) spacing of the enclosure bag from the patient's skin as well. With that framework on the outside of the enclosure bag there is less likely to be any irritation or contamination of the patient's skin because of elimination of skin/bag contact with that enclosure bag.

The invention thus comprises a patient wound treatment arrangement for isolating and enclosing a wound site and minimizing costs and contamination of such treatment to permit pressurized fluid treatment of the wound in the bag. The arrangement comprises an enclosure bag for attachment to a body portion of a patient, the enclosure bag having a first end and a second end, with at least one of the first end or the second end being open; at least one inlet port snorkel arranged through a wall of the enclosure bag; a one way valve arranged on an inner end of each of the inlet port snorkel to permit a wound treating nozzle to be inserted therethrough. A discharge conduit is arranged in fluid communication with the bag, the discharge conduit having a first or upper end and a second or lower end, the discharge conduit having at least one one-way valve therein. The discharge conduit being in communication with a waste collection chamber. The enclosure bag may have a second open end. Each open end of the enclosure bag may have an open end tightener arrangement thereon. The first end of the bag may be wider than the second end of the bag. The discharge conduit may be arranged at a mid-point of the bag to facilitate use of the bag within the field of arthroscopy. The open end of the bag may have a peripheral lip with an adhesive thereon to permit attachment of the bag to the patient at the open end of the bag. The bag may comprise a body enclosure bag for treatment of a hazardous material contaminated patient, the bag having a plurality of inlet snorkels and at least one outlet port in fluid communication with a collection chamber. The enclosure bag may have a support frame arranged outwardly of the bag to permit the bag to be held in a spaced apart orientation from any wounded skin of the patient within the enclosure bag. The enclosure bag may have a textured inner surface. The bag may have a heat resistant material, such as a heat resistant ink placed upon the interior walls of the snorkels of inlet tubes when the walls of the enclosure bag are being heat sealed together, to prevent those portions of those snorkels in line with the heat sealing of the bag from becoming sealed themselves.

A further embodiment comprises use of the snorkels for use in surgical procedures, i.e. laparoscopic surgery

A further embodiment of the present invention includes one side wall of the enclosure bag comprising an extended sheet so as to cover the patient as well as being able to receive a body portion (leg, arm, hand, torso or the like). A yet further embodiment of the present invention comprises an enclosure bag on the side of, and part of a larger body bag or the like to provide a sterile field, or to provide a warmth providing means, and/or to provide protection against contamination and exposure to the elements. Such a “double” bag arrangement may serve to separate body portions from one another, or provide a patient to be attended to by multiple medical personnel without cross contamination or the like. One wall of such tubular body bag or a single (flat/flexible) sheet may preferably comprise one side of the treated-portion enclosure bag and may thus facilitate the cleanliness and/or permit simultaneous multiple treatment modalities upon a patient.

The invention may also comprise a method of treating a wound on a mammalian patient comprising: placing at least a portion of the patient in an enclosure bag through an open end of the enclosure bag; sealing the open end of the enclosure bag onto a wound-free portion of the patient; inserting a wound treatment delivery nozzle through a snorkel inlet in the bag; supplying a wound treatment fluid to the nozzle and spraying the fluid onto the wound of the patient; draining the wound treatment fluid from the bag through a drain conduit into a separate waste collection chamber. The method may include the steps of: maintaining the enclosure bag a spaced distance from the skin of the patient in the enclosure bag; holding the enclosure bag from the wound site by an internal pressure within the enclosure bag; holding the enclosure bag from the wound site by an external frame attached to external portions of the enclosure bag; and wherein the external portions of the enclosure bag comprise any of the snorkel inlet ports.

The invention may also include a method of performing an arthroscopic wound treatment procedure on a joint wound on a mammalian patient comprising the steps of: placing a limb having a joint of the patient in an enclosure bag through an open end of the enclosure bag, the enclosure bag having a first open end and a second open end; sealing the first and the second open ends of the enclosure bag onto a spaced-apart wound-free portion of the patient on either side of the joint of the patient; inserting a wound treatment delivery nozzle through a snorkel inlet in the bag; supplying a wound treatment fluid to the nozzle and spraying the fluid onto the wound of the patient; draining the wound treatment fluid from the bag through a drain conduit into a separate waste collection chamber. The enclosure bag may be is clear and flexible. The enclosure bag may have an inner surface having a texture thereon. The enclosure bag may be pressurized so as to be disposed in a spaced apart manner from the treated skin portion of the patient in the enclosure bag. The enclosure bag may be comprised of a first and a second joined sheet, the second sheet being larger than the first sheet. The second sheet may be comprised of an elongated flexible bag for enclosing a patient, the first sheet and the second sheet defining the body portion enclosure bag.

The enclosure bag may be comprised of a first sheet, and a second larger sheet, the second sheet being larger than the first sheet of flexible material, the sheets being attached to one another about a periphery of the first sheet, the first and second sheets, comprising wall portions of the enclosure bag, second larger sheet also comprising a sterile field defining layer for coverage of a patient. The second larger sheet may be comprised of an elongated enclosure bag to permit enclosure of the body of the patient as well as further separate enclosure of a body portion of the patient being treated.

### BRIEF DESCRIPTION OF THE DRAWINGS

The objects and advantages of the present invention will become more apparent when viewed in conjunction with the following drawings in which:

FIG. 1 is a side elevational view of an enclosure bag in a first embodiment of the present invention;

FIG. 2 is a side elevational view of a enclosure bag of the present invention having both ends open for passage of a patient's limb therethrough, particularly useful for arthroscopic surgery;

FIG. 3 is a side elevational view of an enclosure bag for the treatment of a small wound or eye irrigation in a further embodiment of the present invention;

FIG. 4 is a further embodiment of the present invention for full body enclosure of a patient which may require treatment for hazardous materials or the like; and

FIG. 5 is yet a further embodiment of the bag one side of which comprises a larger sheet for patient coverage or body enclosure.

### DETAILED DESCRIPTION OF THE PREFERRED EMBODIMENTS

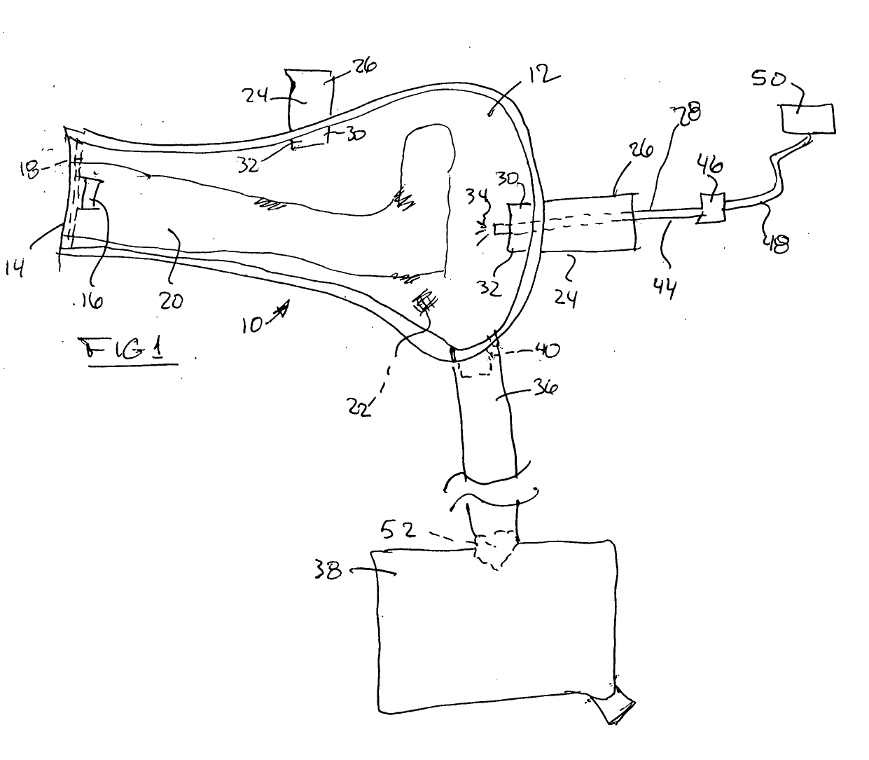

Referring now to the drawings in detail, and particularly to FIG. 1, there is shown the present invention which comprises an elongated, flexible, clear plastic bag 10 having a first, closed, distalmost end 12 and a second, open or proximalmost end 14. In a preferred embodiment of the present invention, the distalmost end 10 is wider than the proximalmost end 14. The distalmost end 12 may be paddle shaped or “omega” shaped so as to comfortably receive a hand or a foot therewithin, as may be seen in FIG. 1. The proximalmost or open end 14 may have for example, a releasable bag-gripping closure tab 16 or a strip of semi-aggressive adhesive 18 circumferentially disposed on an inner facing annular portion thereon, so as to permit the second or proximalmost end 14 to be snugged and brought into close comfortable sealing contact about the patient's arm or leg or body portion 20. The material of the enclosure bag 10 is clear plastic so as to enable the patient's body portion 20 to be readily viewed and treated therewithin.

The enclosure bag 10 preferably has an inner surface which is slightly textured so as to provide a non-smooth surface 22 that won't undesirably grip, stick or adhere to the skin of the patient's limb or body portion 20 during entry or during the treatment procedure. The preferred embodiment of the enclosure bag 10 has a snorkel or inlet tube 24 arranged through a portion thereof at several locations on the enclosure bag 10. Each snorkel 24 preferably comprises a flexible sheet having an outer end 26 for receipt and passage therethrough of a treatment nozzle 28. The snorkel or inlet tube 24 has an inner end 30 which is arranged within the enclosure bag. The inner end 30 of the snorkel 24 may be valve-like such as a duckbill type valve 32, to prevent pressure or treatment fluid 34 from escaping therethrough either during treatment or subsequent to that treatment.

A drainage tube 36 is preferably disposed through a sidewall of the enclosure bag 10 at a lower location thereon, generally opposite to the snorkel inlet tubes 24, as may be seen in FIG. 1. The drainage tube 36 includes an elongated sheath which feeds into a collection chamber 38. The drainage tube 36 preferably has a one way valve 40 at its uppermost end to prevent reversal of flow from the discharge tube 38 or collection chamber 38 back into the body portion enclosure bag 10. The collection chamber 38 may be a flexible bag itself, or a holding tank. The collection chamber 38 may also itself have a discharge valve 42 thereon. The drainage tube or sheath 36 may be sealable after a wound treatment procedure has taken place, by tying the elongated drainage tube 36 into a knot or sealing it with a seal clip or wire.

The treatment nozzle 28 has a proximal end 44 which is manipulable by the attending physician. The proximal end 44 of the treatment nozzle 28 may include a trigger or pressure adjustment arrangement 46 to select pulsed or continuous fluid treatment through the nozzle 28. The proximal end 44 of the treatment nozzle 28 is attached to a fluid pressure supply hose 48 which is in fluid communication with a pressurizable fluid source 50 which provides the pressured treatment fluid(s) 34 into the treatment nozzle 28. The drain tube 36 is preferably arranged on the lowermost side of the enclosure bag 10, generally opposite the snorkels or input tubes 0.24, as shown in FIG. 1.

Treatment gas or air may also be directed through the spray nozzle 28 simultaneously with the fluid 34, or may be directed therethrough subsequent to fluid pressure treatment, to help maintain the enclosure bag 10 as properly spaced apart and out of contact with the patient's skin. The enclosure bag 10 may also be inflated subsequent to the withdrawal of the nozzle 28 to further effect that spacing and separation of the interior surface 22 of the enclosure bag 10 from the patient's wound site and skin. Such an enclosure bag 10 may be left on the patient after initial wound treatment to cover the wound, as a type of wound dressing.

The enclosure bag 10 may also act as an isolation bag in a further embodiment, and be filled with oxygen or other medical treatment fluid/gas. A further embodiment contemplates the bag 10 being filled with a gel for treatment of the wound, or a foam, or hydrogel combination for the wound treatment. Examples of such treatment fluids include hydrogel, silvadene cream, iodine based products or hydrogen peroxide.

A flutter valve or duck bill type valve 52 is also contemplated in a further embodiment, within the collection chamber 38, as shown in FIG. 1. The collection chamber or bag 38 may have gases, liquids, solidifiers or antibiotics added to it (i.e. glutaraldehyde or the like) for sanitation treatment and/or for forming the waste liquid into a gel.

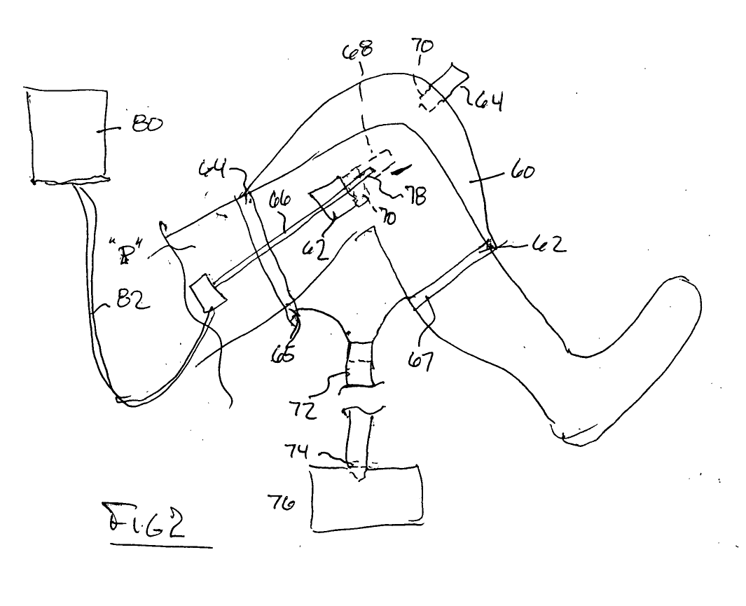

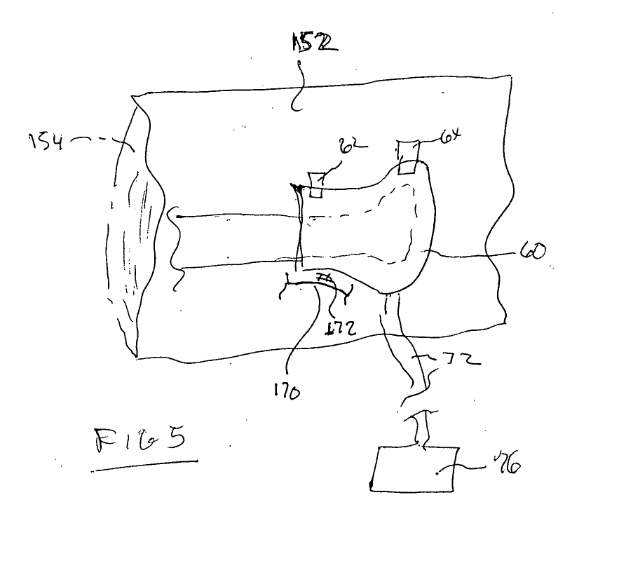

A further embodiment of the present invention comprises a containment or enclosure bag 60 having an open first or distalmost end 62 and an open second or proximalmost end 64 for use, as for example in the field of arthroscopy, as represented in FIG. 2. Both the proximalmost end 64 and the distalmost end 62 would have a sealable annular lip 65 and 67 therearound, for snug attachment to the skin of a patient “P” to permit the viewing and operating on an elbow or a knee or the like, of that patient P. Such an enclosure bag 60 may have side-placed snorkel or inlet tube 62 and an upper or alternate side snorkel tube 64 for placement of a spray nozzle 66 possibly through an endoscope 68 through that/those snorkel/s 62/64 and onto/into a joint of the patient “P” being treated. Such snorkel tubes 62/64 may be in a further embodiment as entry sites for laparoscopic devices, the bag 60 defining a sterile field. The snorkel tubes 62/64 preferably have a duck bill flap valve 70 on its inner side, so as to prevent pressure of fluid from returning out of the snorkel tube 62/64. The snorkel tubes 62/64 may have heat resistant ink on an internal band portion thereof to prevent the snorkel 62/64 from sealing itself together when such a snorkel 62/64 is being heat sealed into the enclosure bag 60. The bag 60 in this embodiment being made from a pair of flexible plastic sheets heat welded about its elongated periphery. One of those sheets 150, as shown in FIG. 5, may be a portion of a larger patient covering protective sheet 152, or as a portion of a larger patient body enclosing bag 154. A collection tube 72 may preferably be arranged at a lowermost pocket in the enclosure bag 60 at a generally midpoint thereof, as shown in FIG. 2. The collection tube 72 would be attached at its distal end 74 to a collection bag or a chamber 76. The nozzle 66 would be pressure adjustable for continuous or pulsed provision of a treatment fluid 78 from a treatment fluid reservoir 80 in communication through a conduit 82 with the proximal end of the nozzle shaft 66. Thus an arthroscopic procedure would be possible without the need for the typically required protective shields and face mask, drains, tubs and subsequent cleansing required procedures of the prior art, while also providing means to collect samples and debris as needed, from the patient's wound.

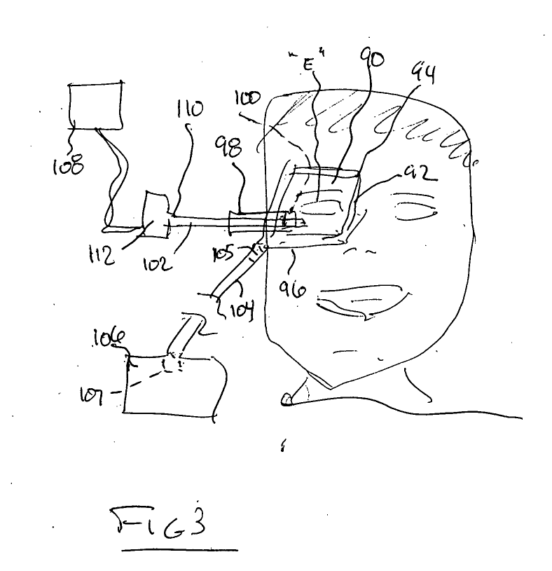

A yet further embodiment of the present invention is contemplated for treatment of smaller body portions such as for the pulsed or continuous fluid treatment and irrigation of a patient's eye, as represented in FIG. 3. Such a treatment would include the use of an enclosure bag 90 having an open first end 92 with a semi aggressive adhesive 94 arranged on its open periphery 96. Such a peripheral adhesive 94 would be disposed about the wound site or the treatment site, for example, such as an eye “E”. Such an enclosure bag 90 would have a snorkel or inlet tube 98 at a central point in the bag 90, as shown in FIG. 3, with a duck bill or flutter bill valve 100 on the inner side of the enclosure bag 90. The spray nozzle 102 would be permitted to be received through the snorkel inlet tube 98 to spray to the wound or the eye “E” within or covered by the bag 90. The enclosure bag 90 would have a drainage tube 104 connected to a collection chamber 106. Such a drainage tube 104 may be cut and sealed at both sides once that cut or breakage in that tube 104 has been made. A one way valve 105, such as a duck bill valve is preferably arranged on the snorkel 98 and a one way valve 107 is also preferably arranged on the upper and the lower ends of the drain tube 104 The spray nozzle or treatment device 102 would have a fluid pressurized source 108 feeding the proximal end 110 of the pressure feed control handle 112 on the nozzle 102 to permit spraying and manipulation by the attending physician. Subsequent to gravity drainage of the irrigation and treatment fluid from the enclosure bag 90 and the sealing of the drainage tube 104, and removal of the nozzle 102 from the snorkel 98, the enclosure bag 90 may be left on the treatment site for subsequent use with further medical treatment fluids, medicaments or wound or eye protection.

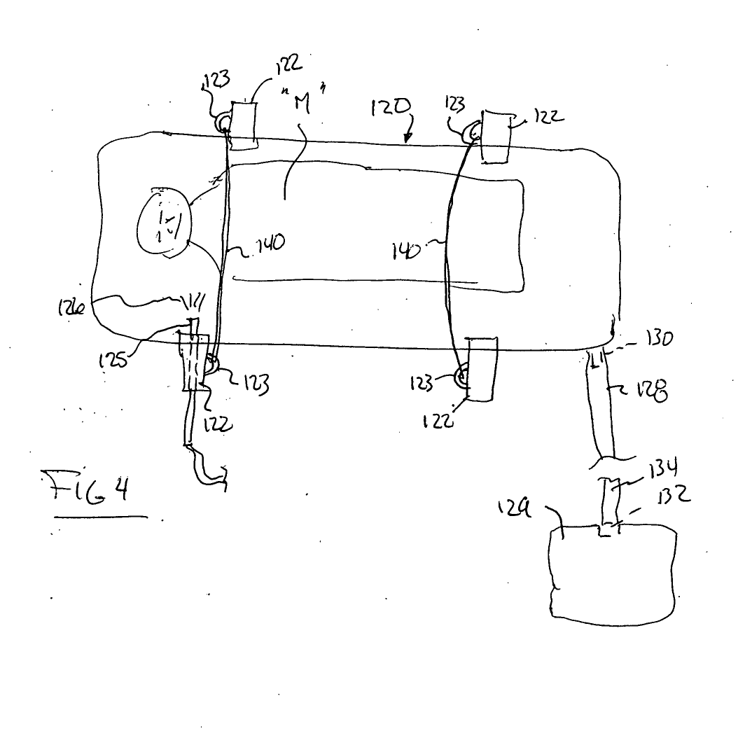

A further embodiment of the present invention is contemplated as a body enclosure bag 120 wherein an “entire” patient “M” is being treated for a hazardous material contamination, is represented in FIG. 4. Such a body containment bag 120 would have a plurality of snorkel inlet tubes or ports 122 for the application of treatment gases 126 supplied from a proper pressurized source, not shown, through a manipulable, port-sealed spray nozzle 125 for the patient therewithin as well as at least one lowermost drainage tube 128 for the passage of the hazardous material into a closed waste containment chamber 129. The drainage tube 128 would preferably have a one way valve 130 in its upper or proximal end, and a one way valve 132 in its lower or distalmost end 134. In such an instance, at least one of those snorkels or inlet tubes 122 may be utilized to provide a constant pressure of treatment gas to keep the enclosure bag away from the surface or skin of the patient “M” therewithin. One or more of the other snorkel inlet tubes 122 may be utilized for the injection of the treatment spray or fluid by the attending medical personnel for the cleansing of the patient, the patient's wounds or washing of hazardous contamination thereof and subsequent closed system collection of that collected hazardous material into the collection chamber 129. Such treatment may be utilized for burn victims, for patients in nursing homes, for treatment of animals by veterinarians, by ambulance personnel and by military personnel or home care personnel.

Such procedures of utilization of the present invention as an enclosure bag eliminates contamination for the medical personnel attending the patient. It eliminates the whirlpool or Sitzbath™ and is a safer way to do pulse or mechanical irrigation of a wound. Those snorkels or tubes provide the access ports for the various spray nozzles and also may act as handles to permit the gripping thereof and manually keep the bag spaced from the patient's skin if pressure within the bag is insufficient or undesired.

In a further preferred embodiment, an outer skeletal frame or support wire 140 of a generally U-shaped configuration may be utilized over the outer surface of the enclosure bag 120 to connectively engage one or more of the snorkel access ports 122 through a tab means 123 thereon to permit the spacing and support the attached enclosure bag 120 and effect that non-pressurized (or assist the pressurized) spacing of the enclosure bag 120 from the patient's skin as well. With that frame 140 on the outside of the enclosure bag 120, there is less likely to be any irritation or contamination of the patient's skin because of elimination of skin/bag contact with that enclosure bag 120.

A further embodiment is partially shown in FIG. 5, wherein an outer bag 170 may be arranged about the first enclosure bag 60, and the annular space therebetween filled with a hardenable resin or foam 172, by injection or the like to function as a splint for the body part therein. The outer bag 170 acting as a mold and protective container for such splint creating resin.

Thus what has been shown is a unique enclosure arrangement comprising a closed system for the cleansing and treatment of a wound or an entire an entire patient while minimizing the time, cost, contamination and danger associated with the treatment systems of the prior art.

We claim:

1. A patient wound treatment arrangement for isolating and enclosing a wound site and minimizing costs and contamination of such treatment to permit pressurized fluid treatment of said wound in said bag, comprising:
an enclosure bag for attachment to a body portion of a patient, said enclosure bag having a first end and a second end, with at least one of said first end or said second end being open;
at least one inlet port snorkel arranged through a wall of said enclosure bag;
a one way valve arranged on an inner end of each of said inlet port snorkel to permit a wound treating nozzle to be inserted therethrough.

2. The patient wound treating arrangement as recited in claim 1, including:
a discharge conduit arranged in fluid communication with said bag, said discharge conduit having a first or upper end and a second or lower end, said discharge conduit having at least one one-way valve therein.

3. The patient wound treating arrangement as recited in claim 2, wherein said discharge conduit is in communication with a waste collection chamber.

4. The patient wound treating arrangement as recited in claim 1, wherein said enclosure bag has a second open end.

5. The patient wound treating arrangement as recited in claim 1, wherein each open end of said enclosure bag has an open end tightener arrangement thereon.

6. The patient wound treating arrangement as recited in claim 1, wherein said first end of said bag is wider than said second end of said bag.

7. The patient wound treating arrangement as recited in claim 4, wherein said discharge conduit is arranged at a mid-point of said bag to facilitate use of said bag within the field of arthroscopy.

8. The patient wound treating arrangement as recited in claim 1, wherein said open end of said bag has a peripheral lip with an adhesive thereon to permit attachment of said bag to said patient at said open end of said bag.

9. The patient wound treating arrangement as recited in claim 1, wherein said bag comprises a body enclosure bag for treatment of a hazardous material contaminated patient, said bag having a plurality of inlet snorkels and at least one outlet port in fluid communication with a collection chamber.

10. The patient wound treating arrangement as recited in claim 1, wherein said enclosure bag has a support frame arranged outwardly of said bag to permit said bag to be held in a spaced apart orientation from any wounded skin of said patient within said enclosure bag.

11. The patient wound treating arrangement as recited in claim 1, wherein said enclosure bag has a textured inner surface.

12. The patient wound treatment arrangement as recited in claim 1, wherein said enclosure bag is comprised of a first sheet, and a second larger sheet, said second sheet being larger than said first sheet of flexible material, said sheets being attached to one another about a periphery of said first sheet, said first and second sheets comprising wall portions of said enclosure bag, second larger sheet also comprising a sterile field defining layer for coverage of a patient.

13. The patient wound treatment arrangement as recited in claim 12, wherein said second larger sheet is comprised of an elongated enclosure bag to permit enclosure of the body of the patient as well as further separate enclosure of a body portion of the patient being treated.

14. A method of treating a wound on a mammalian patient comprising:
placing at least a portion of said patient in an enclosure bag through an open end of said enclosure bag;
sealing said open end of said enclosure bag onto a wound-free portion of said patient;
inserting a wound treatment delivery nozzle through a snorkel inlet in said bag;
supplying a wound treatment fluid to said nozzle and spraying said fluid onto said wound of said patient;
draining said wound treatment fluid from said bag through a drain conduit into a separate waste collection chamber.

15. The method as recited in claim 14, including:
maintaining said enclosure bag a spaced distance from the skin of said patient in said enclosure bag.

16. The method as recited in claim 15, including:
holding said enclosure bag from said wound site by an internal pressure within said enclosure bag.

17. The method as recited in claim 15, including:
holding said enclosure bag from said wound site by an external frame attached to external portions of said enclosure bag,

18. The method as recited in claim 17, wherein said external portions of said enclosure bag comprise any of said snorkel inlet ports.

19. A method of performing an arthroscopic wound treatment procedure on a joint wound on a mammalian patient comprising:
placing a limb having a joint of said patient in an enclosure bag through an open end of said enclosure bag, said enclosure bag having a first open end and a second open end;
sealing said first and second open ends of said enclosure bag onto a spaced apart wound-free portion of said patient on either side of said joint of said patient;
inserting a wound treatment delivery nozzle through a snorkel inlet in said bag;
supplying a wound treatment fluid to said nozzle and spraying said fluid onto said wound of said patient;
draining said wound treatment fluid from said bag through a drain conduit into a separate waste collection chamber.

20. The method as recited in claim 19, wherein said enclosure bag is clear and flexible.

21. The method as recited in claim 19, wherein said enclosure bag has an inner surface having a texture thereon.

22. The method as recited in claim 19, wherein said enclosure bag is pressurized so as to be disposed in a spaced apart manner from the treated skin portion of said patient in said enclosure bag.

23. The method as recited in claim 19, wherein said enclosure bag is comprised of a first and a second joined sheet, said second sheet being larger than said first sheet.

24. The method as recited in claim 23, wherein said second sheet comprises an elongated flexible bag for enclosing a patient, said first sheet and said second sheet defining said body portion enclosure bag.
